(19) United States
(12) Patent Application Publication
(10) Pub. No.: US 2025/0081910 A1
(43) Pub. Date: Mar. 13, 2025

(54) METHOD OF USING/APPLYING A KERATIN HYDROLYSIS PEPTIDE SOLUTION UPON COTTON TO ENHANCE DROUGHT-TOLERANCE

(71) Applicant: CH Biotech R&D Co., Ltd., Nantou City (TW)

(72) Inventors: Jenn Wen HUANG, Taiching (TW); Yi-Chiao CHAN, Keelung (TW); Meng-Ying LI, Nantou (TW)

(73) Assignee: CH Biotech R&D Co., Ltd., Nantou City (TW)

(21) Appl. No.: 18/537,395

(22) Filed: Dec. 12, 2023

(30) Foreign Application Priority Data
Sep. 8, 2023 (TW) 112134395

(51) Int. Cl. A01H 1/00; C05G 3/00; C07K 1/12; C07K 14/465; C07K 14/47; G01N 33/68

(52) U.S. Cl. CPC A01H 1/1225; C05G 3/00; C07K 1/12; C07K 14/465; C07K 14/4741; G01N 33/6848; G01N 2333/465; G01N 2333/4742

(57) ABSTRACT

Present invention teaches the method of using a keratin hydrolysis peptide (“KHP”) solution to enhance the drought-tolerance of the cotton plant. By selectively choosing specific weights of feathers and water, and treating the mixture, though one embodiment does not have water mixed in, to a high-temperature high-pressure hydrolysis process, the resulting solution is confirmed to contain at least 253 peptides and then applied to the cotton seeds and the soil around the cotton plants. Optionally, the KHP solution can be diluted by water, as taught in the specification, before applying to the seeds and the soil as taught herein.

### PRIORITY CLAIM TO FOREIGN APPLICATION

Applicant hereby makes priority claim to a Taiwan application, number 112134395, having the Taiwan filing date of Sep. 8, 2023.

### BACKGROUND OF THE INVENTION

Present invention disclosed and claimed the method and application of a keratin hydrolysis peptide (“KHP”) solution to a cotton plant, known as Gossypium Hirsutum, that will enhance the drought-tolerance of the cotton plant.

The KHP solution is made by a hydrolysis process using feathers and water, with one version of the solution using feathers only, via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Some form of chemical process is used to break down the large protein molecule of the hydrolyzed keratin so that it can penetrate the hair cuticles and do its magic. It is also known that such chemically derived KHP solution was also experimented on farming/crops, though many farmers frown upon such chemical option.

The cotton plant, known as Gossypium Hirsutum, is one of the most important economic crops in the world. The annual production of cotton is approximately 24-28 million metric tons, with the top producing countries being China, India, U.S.A., Brazil, Pakistan, Australia, etc.

To obtain ideal growth and production yield of cotton, cotton needs proper amount of water, sun light, soil nutrients and suitable temperature ranges. Cotton plants generally go through the growth stages of germination, seedling, leaf/canopy development, flowering and maturation. The water is needed for the proper growth of cotton plants in every stage. If there is lack of sufficient water, or even when there is drought, the cotton plant would suffer various illness and/or mal-nutrition symptoms such as wilting, lack of budding, stunt growth, etc.

As the earth is experiencing climate changes, the adversary conditions likely to cause injuries to cotton also become more pronounced. One of the major concerns is the impact of drought that would negatively affect the growth, and thus the production, of cotton.

Experts from the “Cotton 2040” advocacy, part of the “Forum for The Future” global non-profit organization, have repeated pointed out the danger of reduced cotton production due to drought.

In light of the impending global crisis, the inventors of present application experimented and worked on the method of employing a high-temperature and high-pressure hydrolysis process to break down the keratin in feathers, and to form a hydrolyzed peptide solution that can be applied to cotton (from seeds to plants) that will help with the drought-tolerance of cotton.

### SUMMARY OF THE INVENTION

Given the known crisis as stated above, there are existing technologies that can be used to counter the adversary conditions faced by cotton. For example, a Chinese patent, CN102604961A disclosed a certain genetic-modification technique by tweaking the TT1 gene to express better anti-adversity attributes. There are other technical developments that work in similar fashion to adjust certain protein and/or genetic compositions of cotton for better survivability in adversary conditions.

Such technique, however, is limited in value because that the adjustment/tweaking in the genetic composition/expression is pretty expensive and is less useful when the exact adverse conditions change.

The inventors of present invention chose not to adopt the expensive route, and instead by creating a keratin solution and method of applying the solution to the cotton in the regular growth cycles in the normal course of farming activities, thus is a workable remedy to deal with drought and helpful to more people in a more cost-effective manner.

The keratin solution is primarily based upon feather, which contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of α-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application, through experiments, selected different feather and water compositions and did the hydrolysis at higher temperature and higher pressure setting, and obtained a different keratin hydrolysis peptide (“KHP”) solution that can be used on different crops/plants.

The inventors further experimented the method of applying the KHP solution, some with further dilution, in the field, on the cotton plant, as well as other plants and proved the effectiveness of the method in combating the injuries or symptoms that may be caused by drought.

By making the KHP solution and applying to the cotton plants as disclosed herein, it is

found that, even where the cotton plants have gone through periods of drought during the growth stages, they show stronger signs of recovery as compared to cotton plants that are not treated, or where the soil is not infused with the solution.

The first embodiment of present invention uses a mixture of water and feathers (water content 50%) at the weight ratio of 5:4 (specific weight of 50 kg water and 40 kg feathers) and subject the mixture to a thermal hydrolysis process (80 minutes) to create a solution, resulting in the solution having molecular mass of 593.3-3,508.9 Dalton and containing at least 253 peptides as confirmed.

The inventors of present application used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The solution then is applied, with some dilution and with optional mixing of nitrogen-based fertilizer, to the cotton plants.

A second embodiment uses only feathers (water content 46%), specific weight of 70 kg, then treat it to a thermal hydrolysis process (40 minutes), resulting in a solution with molecular mass of 705.9-3,194.7 Dalton, containing at least 253 peptides as confirmed by the mass spectrometer and referencing the BIOPEP-UWM database.

The same method of application to the cotton as in first embodiment can also be used on the second embodiment

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

Table II shows the parameters of the KHP solutions for the two embodiments.

FIG. 1 shows the comparisons of cotton plants re growth status measured by the shoot dry weight.

FIG. 2 shows the comparison of cotton plants re growth status measure by foliage strength.

### DETAILED DESCRIPTION OF THE INVENTION

The keratin hydrolysis peptide (“KHP”) solution of present invention, as a first specific embodiment, is made by a high-temperature and high-pressure process to treat a mixture of water and feathers where the weight ratio whereby the weight ratio of water and feathers is set at 4:5. In the disclosure below, the specific weight of 50 kg feathers and 40 kg water will be used.

The hydrolysis process takes the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 3.0×10⁵˜4.5×10⁵ ppm;

The confirmation of the some of the bioactive peptides is further done by referencing the BIOPEP-UWM database.

The solution can then be applied to the soil around the planted cotton.

The method of using a keratin hydrolysis peptide (KHP) solution stated above can be first applied to the cotton seeds before planting.

The method of using a keratin hydrolysis peptide (KHP) solution stated above can take a further dilution step by adding water by volume at the ratio of 1:50-100 and is infused into the sold around the planted cotton.

The method of using a keratin hydrolysis peptide (KHP) solution stated above where the solution is mixed with nitrogen-based fertilizer at the ratio of 1.25:1-2.5:1 for application to the seeds or soil around the planted seeds of cotton.

A method of using the KHP solution of present invention, as a second specific embodiment, is made by a high-temperature and high-pressure process to treat a mixture of feathers without water. In the disclosure below, the specific weight of 70 kg feathers will be used.

The hydrolysis process takes steps of:
a. Preparing the KHP solution by putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 3.0×10⁵˜4.5×10⁵ ppm;

The confirmation of some of the bioactive peptides is further done by referencing the BIOPEP-UWM database.

The solution can then be applied to the soil around the planted cotton.

The method of using a keratin hydrolysis peptide (KHP) solution stated above can first be applied to cotton seeds before planting to the soil.

The method of using a keratin hydrolysis peptide (KHP) solution stated above can diluted with water by volume at the ratio of 1:50-100 and is infused into the sold around the planted cotton.

The method of using a keratin hydrolysis peptide (KHP) solution state above can be further mixed with nitrogen-based fertilizer at the ratio of 1.25:1-2.5:1 for application to the seeds or soil around the planted seeds of cotton.

The inventors of present application conducted field tests on the cotton plants in the field and confirmed the effectiveness of the invention disclosed and claimed herein.

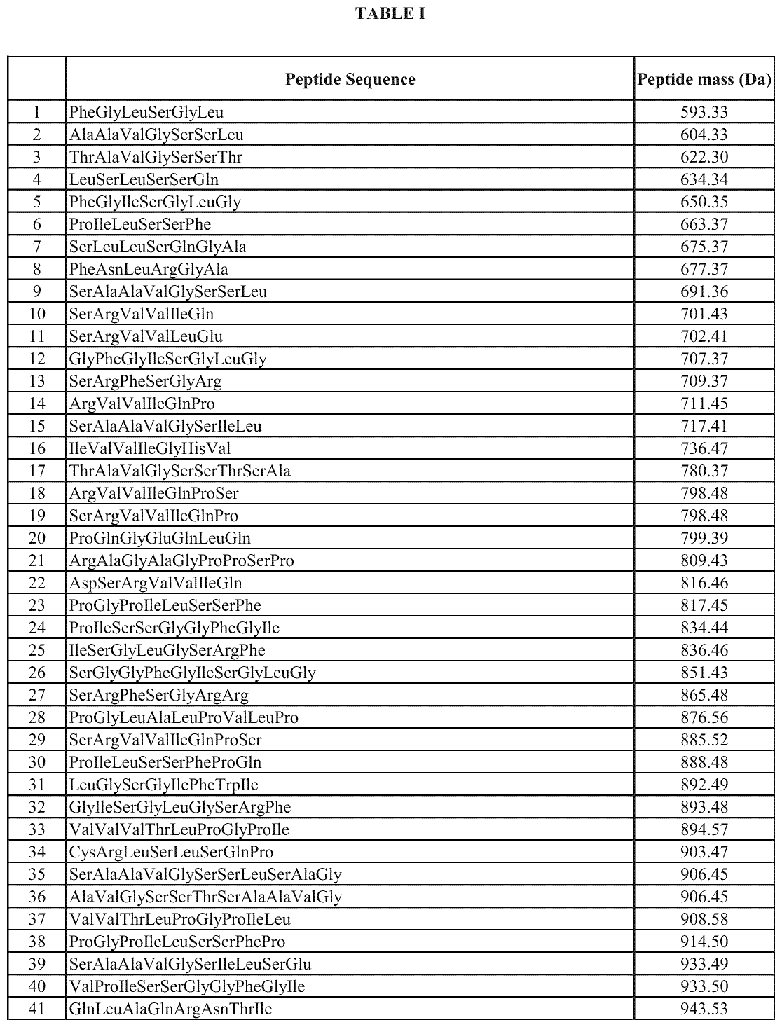

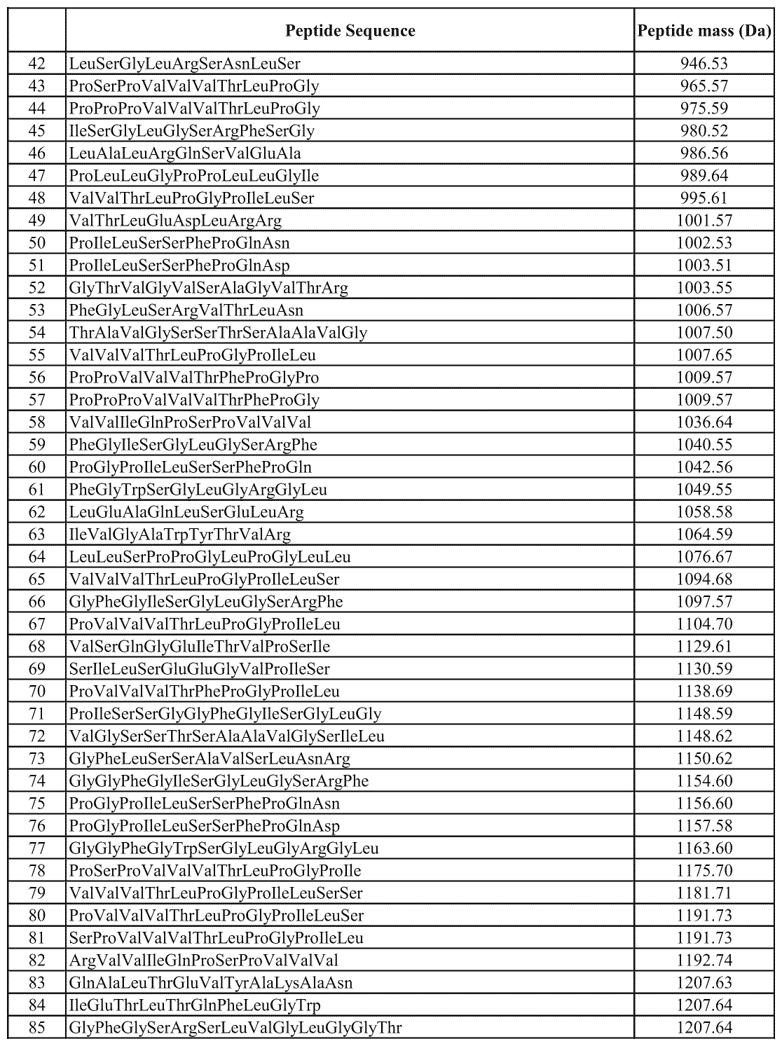

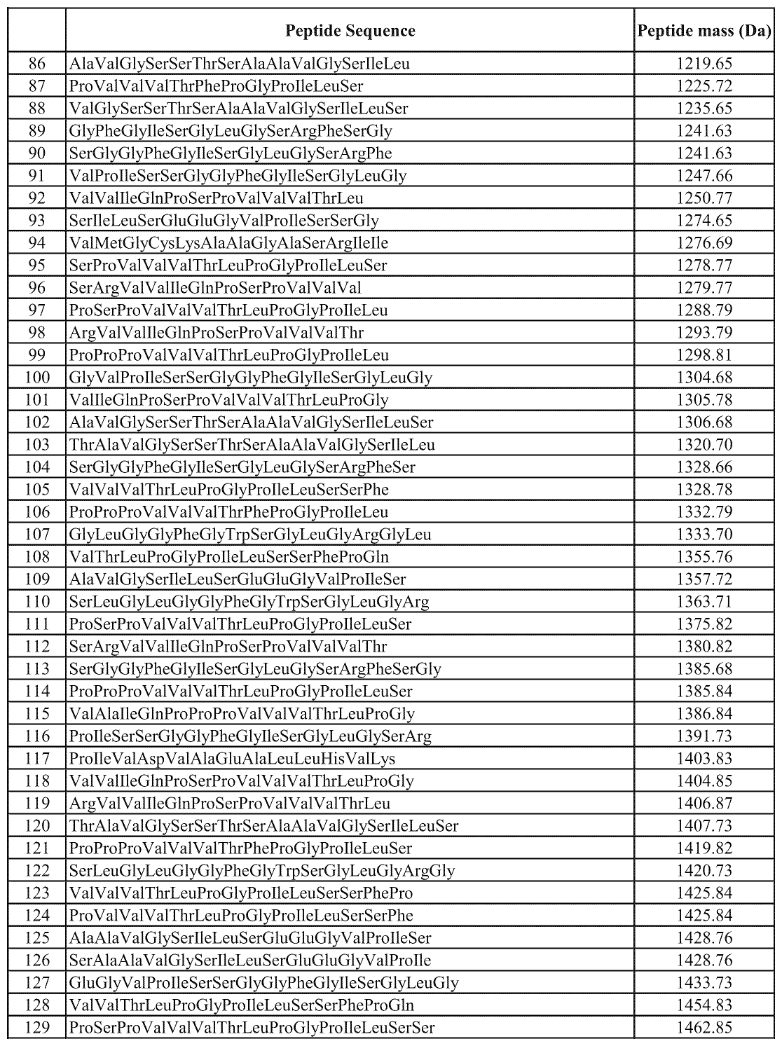

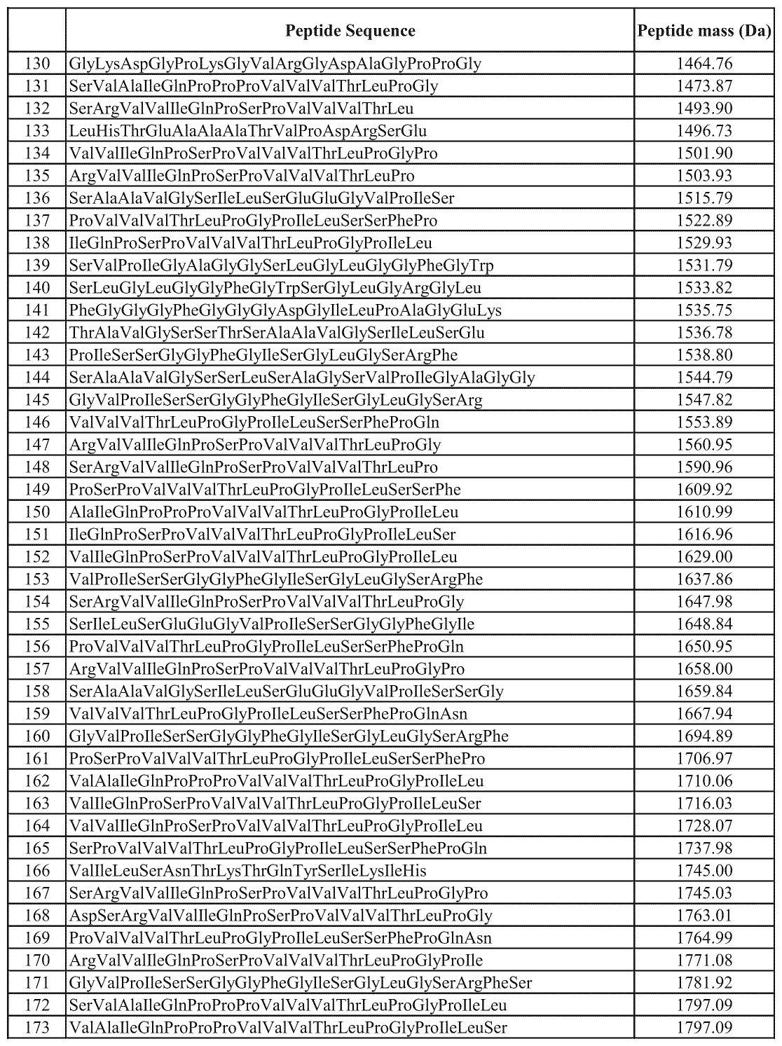

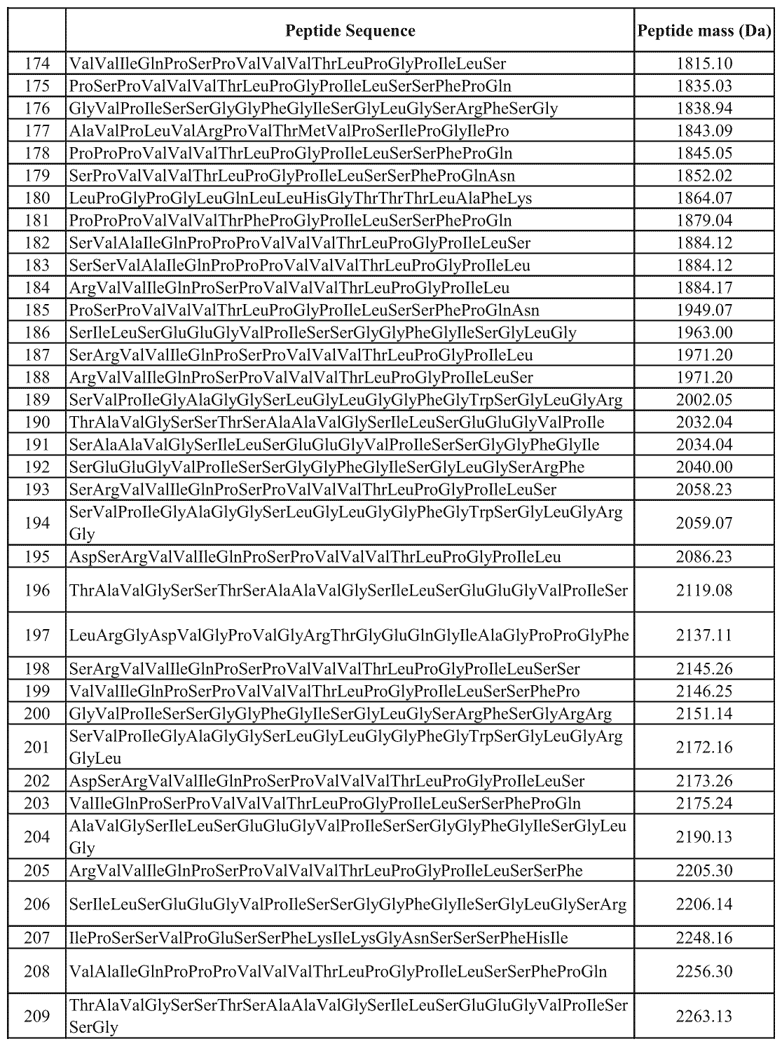

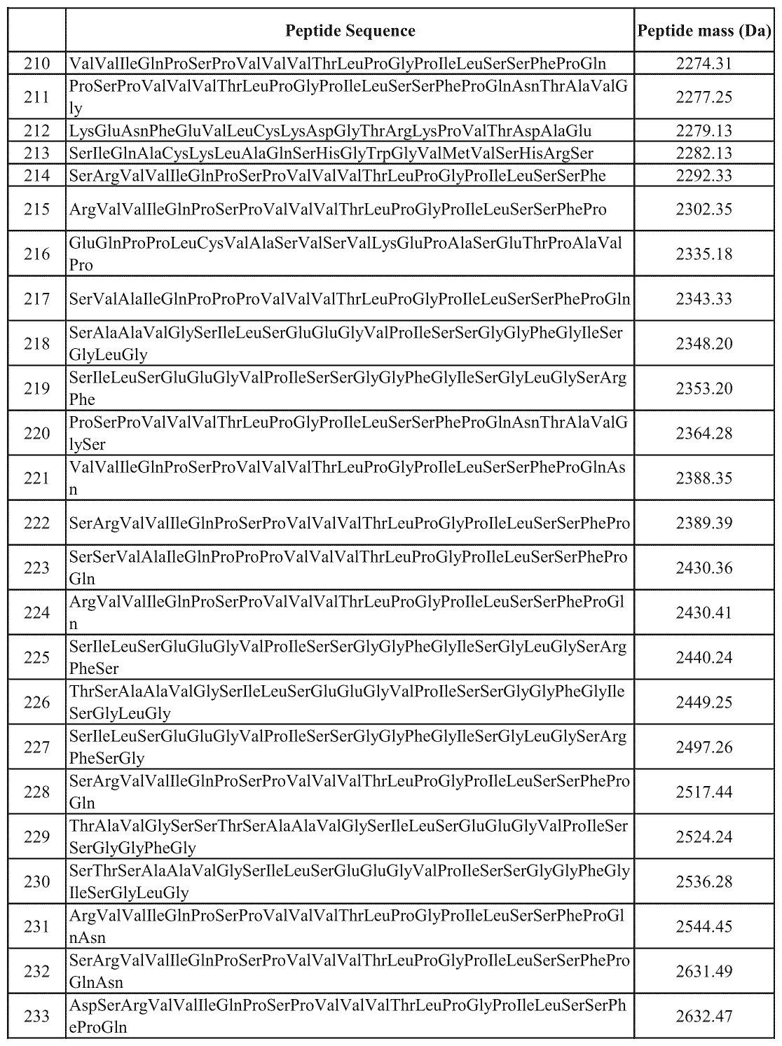

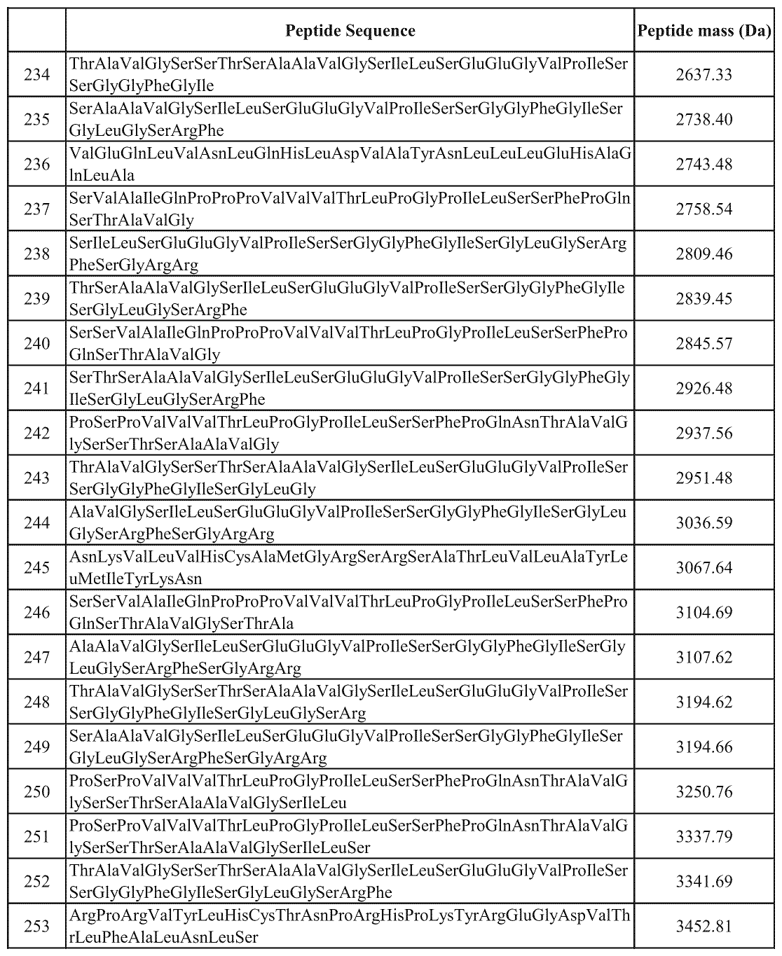

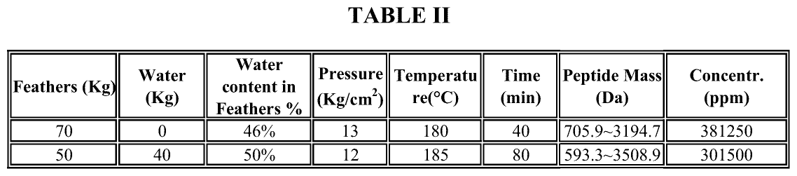

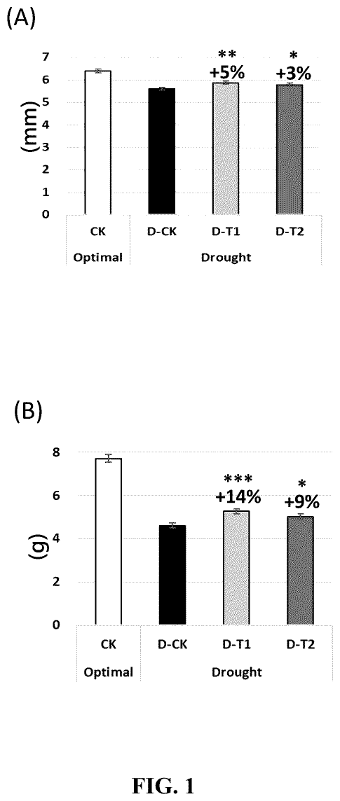

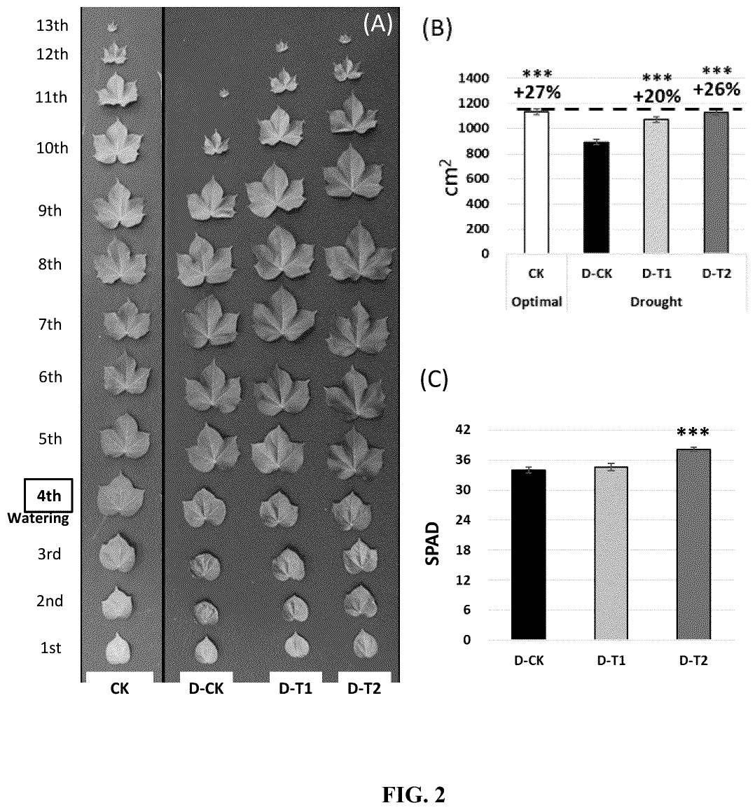

Four sets of cotton plants are compared. Each set has 12 plants. The test results are shown in FIGS. 1 and 2.

The CK group is the cotton plants group receiving normal water. The D-CK group is the cotton plants group suffering drought. The D-T1 and D-T2 are the cotton plants groups receiving the KHP solution treatment.

Every pot of the D-T1 cotton plant is given 3 ml KHP solution diluted 100 times, ending up with 300 ml sprayed to the plant, along with 2.4 g normal fertilizer. Every pot of the D-T2 cotton plant is given 6 ml KHP solution diluted 50 times, ending up 300 ml sprayed to the pant, along with 2.4 g normal fertilizer.

As can be seen in FIG. 1(A), the stem width measurements in the KHP solution treated groups are measured to be 5% and 3% better as compared to the drought group. The FIG. 1(B) measure the dry weight of the shoot; the KHP solution treated groups are measured to be 14% and 9% better.

FIG. 2 shows the comparisons of leaf health status. 2(A) shows the comparisons of the leaf images. FIG. 2(B) compare the leaf area sizes, with the KHP solution treated groups 20% and 26% better. FIG. 2(C) compares the SPAD chlorophyll count. Again, the KHP solution treated groups show better growth status.

The leaf area comparison was done by scanning the leaf surface, then using WinFolia app (WinFOLIA Pro 2014a, Regent Instruments Inc.) to compute the leaf area and analysis. The chlorophyll count is by using the SPAD-520plus, Konica Minolta to measure the chlorophyll amount on the leaf blade.

Further experiment was done by the inventors where some nitrogen-based fertilizer is mixed into the KHP solution for applying to the cotton plants. At the KHP solution vs. nitrogen fertilizer ratio of 1.25:1-2.5:1, the nitrogen-mixed solution also works well for the drought-tolerance as disclosed herein.

Overall, the research and experiment show that the method of making the KHP solutions and the applications substantially add to the drought-tolerance of the cotton plant, as intended by the disclosure of the present application. Such method will no doubt be well-received in the cotton farming and growing industry to help counter the adverse environmental factors as discussed hereinabove.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to a cotton plant for the enhancement of the plant's drought tolerance, comprising the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 3.0×10⁵˜4.5×10⁵ ppm;
d. applying the solution to the soil around cotton plants.

2. The method of using a keratin hydrolysis peptide (KHP) solution of claim 1 where the solution is first applied to the seed of cotton plants before planting to soil.

3. The method of using a keratin hydrolysis peptide (KHP) solution of claim 1 where the solution is diluted with water by volume at the ratio of 1:50-100 and is infused into the sold around the planted cotton.

4. The method of using a keratin hydrolysis peptide (KHP) solution of claim 1 further is mixed with nitrogen-based fertilizer at the ratio of 1.25:1-2.5:1 for application to the seeds or soil around the planted seeds of cotton.

5. A method of using a keratin hydrolysis peptide (KHP) solution to a cotton plant for the enhancement of the plant's drought tolerance, comprising the steps of:
a. Preparing the KHP solution by putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 3.0×10⁵˜4.5×10⁵ ppm;
d. applying the solution to the soil around cotton plants.

6. The method of using a keratin hydrolysis peptide (KHP) solution of claim 5 where the solution is first applied to the seeds before planting into soil.

7. The method of using a keratin hydrolysis peptide (KHP) solution of claim 5 where the solution is diluted with water by volume at the ratio of 1:50-100 and is infused into the sold around the planted cotton.

8. The method of using a keratin hydrolysis peptide (KHP) solution of claim 5 further is mixed with nitrogen-based fertilizer at the ratio of 1.25:1-2.5:1 for application to the seeds or soil around the planted seeds of cotton.
